(19) United States
(12) Patent Application Publication
(10) Pub. No.: US 2026/0056200 A1
(43) Pub. Date: Feb. 26, 2026

(54) METHOD FOR IMMUNODETECTION USING ANTIBODY COCKTAIL

(71) Applicant: IMAM ABDULRAHMAN BIN FAISAL UNIVERSITY, Dammam (SA)

(72) Inventors: Omar Soliman Mohamed ELMASRY, Dammam (SA); Lamya Zohair YAMANI, Dammam (SA); Khaldoon Mohammed A. ALSAMMAN, Dammam (SA)

(73) Assignee: IMAM ABDULRAHMAN BIN FAISAL UNIVERSITY, Dammam (SA)

(21) Appl. No.: 18/812,963

(22) Filed: Aug. 22, 2024

(51) Int. Cl. G01N 33/574; G01N 33/561

(52) U.S. Cl. CPC G01N 33/57484; G01N 33/561

(57) ABSTRACT

A method of detection of one or more antigens using a cocktail of one or more primary and secondary antibodies in a single step using a non-conventional Western blot protocol. Various antibody cocktails that can be used in the immunodetection methods.

### p BACKGROUND

### Technical Field

The present disclosure is directed to a method for immunodetection, particularly a method for detecting target proteins using a mixture of primary and secondary antibodies.

### Description of Related Art

The “background” description provided herein is for the purpose of generally presenting the context of the disclosure. Work of the presently named inventors, to the extent it is described in this background section, as well as aspects of the description which may not otherwise qualify as prior art at the time of filing, are neither expressly nor impliedly admitted as prior art against the present disclosure.

The structural basis of antigen-antibody recognition lies in the functionality of antibody paratopes and their ability to specifically bind to antigens via antigenic determinants, also known as epitopes. Within the variable region of antibodies, there are six hypervariable loops, known as complementary determining regions (CDRs), responsible for the specificity towards antigen recognition and binding. The association of antibodies with antigens happens through various non-covalent interactions that play a role in the high specificity and affinity that is used for reversible binding. This interaction does not only play a role in immunity, but it also may be applied as an experimental tool in diagnostics and biomedical research. The interaction may also be exploited for therapeutic approaches.

Antigen-antibody interactions may be divided into two types; in vivo and in vitro, the former occurring under natural conditions while the latter happens under artificial conditions. Among the recognized in vitro methods applied is the Western blot technique used in the immunodetection of proteins. It is a simple and powerful technique used to investigate and study protein-protein interactions by identifying presence, relative mass, and abundance. Moreover, it may also be useful when examining the presence of relevant post-translational modifications at protein levels. The inception of this protocol relied on specific interactions of antibodies towards target antigens by separating the sample mixture via electrophoretic transfer based on differences in molecular weight of each protein. Once these proteins were separated on the gel, they were transferred to a membrane where they encounter an incubation period with a blocking reagent to minimize non-specific binding. After the blocking, primary antibodies were added to probe the target protein. Further incubation and washing steps were performed, followed by adding secondary antibodies conjugated with labeled chemiluminescent molecules to provide signal detection via densitometry analysis of the specific probed protein bands. In this technique, primary and secondary antibodies are added at different steps, each binding specifically to their intended counterparts. Primary antibodies specifically bind to proteins on the membrane because they can directly bind to antigens of the target protein, thereby not requiring to be pre-adsorbed. The secondary antibody is unable to bind to the target protein but interacts with the heavy chains of the primary antibody that has been previously bound to the target protein. It is known that secondary antibodies are used for immunolabeling rather than initial antigen-antibody binding. This secondary antibody may need to be pre-adsorbed, allowing minimal non-specific binding and increased binding specificity.

The complete Western blot protocol involves multiple interdependent steps that may be considered complex and tedious. When the time comes for protocol adaptation, expertise and subjective choices are influenced by those interdependent steps. Accordingly, such subjectivity contributes to the reproducibility of obtained results and may become a source of error due to the variations made while performing the methodology. As previously mentioned, a concern of Western blotting is the complex and tedious protocol. A method of co-incubating the primary and secondary antibodies with the membrane instead of being added at different times has been suggested to reduce the number of steps and incubation periods within the protocol; however, it is believed that Western blotting procedures using this method may produce false negative results due to the prozone effect. The prozone effect is an interference caused by high antibody titers, inhibiting antigen-antibody lattice formation that should be observed when both antigen and antibodies are present at optimal levels. Therefore, this effect is presumed to disrupt the interaction between the primary antibody and antigens by masking the actual presence of the antigens on the blot.

A need remains to devise a method of immunodetection in which combining and co-incubating primary and secondary antibodies together shows no effect on target protein band density. Accordingly, an object of the present disclosure is to provide a method wherein both primary and secondary antibodies can be used together in a mixture for the detection of antigens effectively and efficiently, similar to having them added and incubated in separate steps.

SUMMARY

In an exemplary embodiment, a method of immunodetection is described. The method comprises mixing a protein lysate comprising a target protein and a reaction buffer to form a reaction mixture; heating the reaction mixture for 1 to 10 minutes at a temperature of 90 to 100 ° C.; and loading the reaction mixture in a well of a gel in an electrophoresis chamber. The electrophoresis chamber comprises a Sodium Dodecyl Sulphate-Polyacrylamide Gel Electrophoresis (SDS-PAGE) running buffer. The method further includes running gel electrophoresis on the gel at 80 to 120 V for 30 to 90 minutes; rinsing the gel with a transfer buffer; transferring blot bands from the gel to a polyvinylidene fluoride membrane; contacting the polyvinylidene fluoride membrane with a first blocking buffer for 20 to 120 minutes at a temperature of −10 to 15° C.; and contacting the polyvinylidene fluoride membrane with a second blocking buffer for 40 to 80 minutes at a temperature of −10 to 15° C. The second blocking buffer comprises an antibody cocktail, wherein the antibody cocktail comprises a primary antibody and a secondary antibody. The primary antibody is selected from a group consisting of mouse monoclonal antibody to β-actin, anti-TAP-1 antibody, rabbit polyclonal antibody to GAPDH, and mouse monoclonal antibody to lamin B1 and the secondary antibody is selected from a group consisting of goat anti-mouse HRP conjugate and goat anti-rabbit HRP conjugate. The method further includes washing the polyvinylidene fluoride membrane with a wash buffer; incubating the polyvinylidene fluoride membrane with a mixture of detection reagents for 30 to 90 seconds; and imaging the polyvinylidene fluoride membrane to detect the target protein.

In some embodiments, the second blocking buffer comprises a primary antibody and a secondary antibody in a ratio of 0.1:1 to 10:1.

In some embodiments, the reaction buffer comprises a 2× Laemmli buffer and β-mercaptoethanol in a volumetric ratio of 10:1 to 25:1.

In some embodiments, a volumetric ratio of the protein lysate to the reaction buffer is from 1:1 to 1:20.

In some embodiments, the gel is a mini-protean TGX precast gel.

In some embodiments, the SDS-PAGE running buffer comprises a 10 × tris/glycine/sodium dodecyl sulfate buffer and water in a volumetric ratio of 1:5 to 1:15.

In some embodiments, the transferring occurs using a sandwich transfer process with a transfer-blot turbo transfer system for 20 to 30 minutes.

In some embodiments, the first blocking buffer comprises a 2 to 8% wt./v milk powder and 1× TBST solution.

In some embodiments, the primary antibody has a final working dilution of 1:800 to 1:1200.

In some embodiments, the secondary antibody has a final working dilution of 1:2500 to 1:3500.

In some embodiments, the mixture of detection reagents is a Clarity Western peroxide reagent and a Clarity Western luminol/enhancer reagent.

In some embodiments, the target protein is at least one selected from a group consisting of β-actin, GAPDH, p62, AMPK, CHOP, JNK, and TAP-1.

In some embodiments, the target protein is detected in cells in a group consisting of OVCAR4, PEO4, PEO6, SKOV3, and HCT-116.

In some embodiments, the antibody cocktail is stable for at least one year at a temperature of −30 to −10° C.

In some embodiments, the method of immunodetection further comprises removing cells comprising the target protein from a culture; mixing the cells comprising the target protein with a RIPA lysing and extraction buffer to form a first mixture; centrifuging the first mixture for 1 to 10 minutes at a speed of 10,000 to 15,000 rpm; and decanting a supernatant to form the protein lysate.

In some embodiments, the method of immunodetection further comprises adding phosphatase inhibitors and protease inhibitors to the first mixture.

In some embodiments, the antibody cocktail is a single reagent.

In some embodiments, the secondary antibody is conjugated to the primary antibody before the primary antibody and the second primary antibody is conjugated to the target protein.

In some embodiments, the target protein is a mixture of at least two proteins.

In some embodiments, the secondary antibody is conjugated with a signal generator.

These and other aspects of the non-limiting embodiments of the present disclosure will become apparent to those skilled in the art upon review of the following description of specific non-limiting embodiments of the disclosure in conjunction with the accompanying drawings. The foregoing general description of the illustrative embodiments and the following detailed description thereof are merely exemplary aspects of the teachings of this disclosure and are not restrictive.

### BRIEF DESCRIPTION OF THE DRAWINGS

A more complete appreciation of the present disclosure (including alternatives and/or variations thereof) and many of the attendant advantages thereof will be readily obtained as the same becomes better understood by reference to the following detailed description when considered in connection with the accompanying drawings, wherein:

FIG. 1 is a flowchart depicting a method of immunodetection, according to certain embodiments.

FIG. 2A depicts a potential antibody cocktail containing primary and secondary antibodies for single-step detection, according to certain embodiments.

FIG. 2B depicts a potential antibody cocktail containing two primary antibodies and a secondary antibody for multiplex detection, according to certain embodiments.

FIG. 3A illustrates a traditional (conventional) protocol for immunodetection, according to certain embodiments.

FIG. 3B illustrates an improved (non-conventional) protocol (method of the present disclosure) for immunodetection, according to certain embodiments.

FIG. 4A shows band densities of β-actin in the improved protocol (method of the present disclosure) and the traditional Western blotting protocol, according to certain embodiments.

FIG. 4B shows band densities of transporter associated with antigen processing 1 (TAP-1) in the improved protocol (method of the present disclosure) and the traditional Western blotting protocol, according to certain embodiments.

FIG. 4C shows band densities of glyceraldehyde 3-phosphate dehydrogenase (GAPDH) in the improved protocol (method of the present disclosure), according to certain embodiments.

FIG. 5 compares the mean rank of band densities of β-actin in the improved protocol (method of the present disclosure) and the traditional Western blotting protocol, according to certain embodiments.

FIG. 6 shows a blot for co-detection of β-actin and GAPDH proteins in different ovarian cancer cell types, according to certain embodiments.

FIG. 7 shows a blot for co-detection of β-actin and lamin B1 in different ovarian cancer cell lines, according to certain embodiments.

FIG. 8 compares a blot for detecting β-actin protein in PEO6 cells with a premade antibody cocktail (stored for 72 hours) and simultaneous addition of primary and secondary antibodies, according to certain embodiments.

FIG. 9 shows a blot for co-detection of β-actin and GAPDH (stripped membrane) in OVCAR 4 ovarian cancer cells, according to certain embodiments.

FIG. 10 shows a blot for co-detection of GAPDH and p62 (SQSTM1) (left), and β-actin, GAPDH, and p62 (right) in hematopoietic cell transplantation (HCT) colon cancer cells, according to certain embodiments.

FIG. 11 shows a blot for co-detection of 5′ AMP-activated protein kinase (AMPK), CCAAT-enhancer-binding protein homologous protein (CHOP), and c-Jun N-terminal kinase (JNK) in OVCAR 4 cancer cells (stripped membrane), according to certain embodiments.

FIG. 12 shows a blot for detecting TAP-1 in PEO6 ovarian cancer cells, according to certain embodiments.

### DETAILED DESCRIPTION

In the following description, it is understood that other embodiments may be utilized, and structural and operational changes may be made without departure from the scope of the present embodiments disclosed herein.

Reference will now be made in detail to specific embodiments or features, examples of which are illustrated in the accompanying drawings. In the drawings, wherever possible, corresponding or like reference numerals will be used to designate identical or corresponding parts throughout the several views. Moreover, reference to various elements described herein are made collectively or individually when there may be more than one element of the same type. However, such references are merely exemplary in nature. It may be noted that any reference to elements in the singular may also be construed to relate to the plural and vice-versa without limiting the scope of the disclosure to the exact number or type of such elements unless set forth explicitly in the appended claims. Further, as used herein, the words “a,” “an” and the like generally carry a meaning of “one or more,” unless stated otherwise.

Furthermore, the terms “approximately,” “approximate,” “about,” and similar terms generally refer to ranges that include the identified value within a margin of 20%, 10%, or preferably 5%, and any values therebetween.

As used herein, the term “antigen” refers to a peptide, lipid, moiety, toxin, foreign particulate matter, polysaccharide, allergen, nucleic acid, or any other similar molecule that can bind to a specific antibody or T-cell receptor and is capable of triggering an immune response in the body, particularly the production of antibodies.

As used herein, the term “antibody” refers to proteins produced by the body in response to and counteracting a specific antigen. Antibodies produce an immune reaction, particularly in response to a foreign antigen that has entered the body.

As used herein, the term “monoclonal antibody” (also referred to as mAb and/or moAb) refers to an antibody derived from a lineage of homogeneous antibodies that are highly specific for a particular antigen. Monoclonal antibodies may have monovalent affinity, binding only to the same epitope. A monoclonal antibody may be produced from a cell lineage made by cloning a unique white blood cell. Monoclonal antibodies may be laboratory-produced molecules which are engineered to serve as substitute antibodies that can restore, enhance, modify, or mimic an immune system attack on antigens.

As used herein, the term “epitope” (also known as an antigenic determinant) refers to a part of the antigen recognized by various components of the immune system, including antibodies, B-cells, and T-cells, during an immunogenic response. The part of an antibody that binds to the epitope is called a paratope.

As used herein, a “paratope” (also known as an antigen-binding site) refers to a part of an antibody which recognizes and binds to an antigen. It is found at the tip of the antibody's antigen-binding fragment.

As used herein, the term “immunodetection” refers to a method for detecting specific proteins or antigens using antibodies. Immunodetection may be used to identify specific proteins blotted to membranes.

As used herein, the term “antibody conjugate” refers to an antibody or an antigen-binding fragment that is attached to another entity such as a chemiluminescent material, a label, a drug, a tag, and/or a toxin. The attachment may be via covalent or non-covalent bonding. An antibody conjugate may be a polyclonal or monoclonal antibody that has a molecule attached which can be used to create a detectable signal. Detection may be visualized by color-generation, fluorescence, or other signals.

As used herein, the term “specificity” refers to the ability of an antibody and/or an antigen to differentiate between other antibodies and/or antigens.

As used herein, the term “electrophoresis” refers to a technique to separate protein or nucleic acid molecules based on their size and electrical charge.

As used herein, the term “SDS PAGE” (sodium dodecyl sulfate-polyacrylamide gel electrophoresis) refers to a discontinuous electrophoretic system used to separate proteins, with molecular masses between 5 and 250 kDa, based on their molecular weights.

As used herein, the term “buffer” refers to a solution that can resist pH change after dilution or addition of small quantities of an acid or base.

As used herein, the term “protein lysate” refers to a protein-rich material extracted by lysis of cultured cells.

Aspects of the present disclosure are directed to methods for detecting target proteins using a mixture of primary and secondary antibodies. More particularly, aspects of the present disclosure are directed to immunodetection methods involving Western blot procedures wherein the primary and secondary antibodies are added and incubated with the membrane simultaneously. The results of simultaneously incubating primary and secondary antibodies during Western blotting deliver similar results as in the case where primary and secondary antibodies are added at different steps of the procedure. Experiments were carried out for the detection of single proteins as well as multiple proteins, including β-actin, glyceraldehyde 3-phosphate dehydrogenase (GAPDH), lamin B1, and transporter associated with antigen processing 1 (TAP-1) proteins. The protein band densities for these proteins were analyzed by conventional Western blot protocols and the Western blot protocol of the present disclosure. The results depict similar band densities obtained from both protocols indicating that an antibody cocktail can be utilized for the detection of single as well as multiple proteins in a single run instead of conducting multiple incubation and washing runs. Moreover, the antibody cocktail comprising the primary and secondary antibodies is found to be highly stable and can be made and preserved for long durations of time.

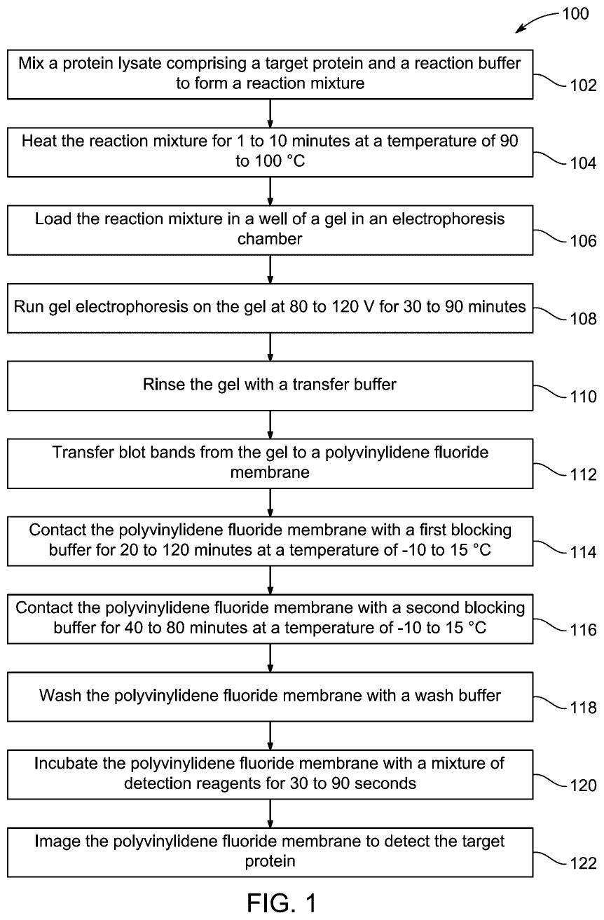

FIG. 1 illustrates a flow chart of a method 100 of immunodetection. The order in which the method 100 is described is not intended to be construed as a limitation, and any number of the described method steps can be combined in any order to implement the method 100. Additionally, individual steps may be removed or skipped from the method 100 without departing from the spirit and scope of the present disclosure.

At step 102, the method 100 includes mixing a protein lysate with a reaction buffer to form a reaction mixture. The protein lysate comprises at least one target protein. In some embodiments, the at least one target protein is extracted from a mammalian cancer cell line. In one embodiment, the at least one target protein is extracted from a human cancer cell line. In another embodiment, the at least one target protein is extracted from a human ovarian cancer cell line. In some embodiments, the at least one target protein is extracted from human ovarian cancer cell lines, wherein the cell lines may be selected from a group consisting of PEO4, PEO6, OVCAR4, and SKOV3. In another embodiment, the at least one target protein is extracted from a mammalian colon cancer cell line. In certain embodiments, the at least one target protein is extracted from a human colon cancer cell line. In some embodiments, the at least one target protein is extracted from a human colon cancer cell line wherein the colon cancer cell line may be HCT-116. The at least one target protein may be extracted from any cell, cell line, tissue, and/or any material containing proteins known in the art.

In some embodiments, the protein lysate includes a mixture of target proteins and a reaction buffer. The number of target proteins can vary, with examples including two or more, three or more, four or more, and five or more target proteins, depending on the specific embodiment. In some embodiments, the at least one or more target proteins may be selected from a group consisting of β-actin, glyceraldehyde-3-phosphate dehydrogenase (GAPDH), p62, 5′ AMP-activated protein kinase (AMPK), C/EBP homologous Protein (CHOP), c-Jun N-terminal kinase (JNK), and transporter associated with antigen processing 1 (TAP-1).

In some embodiments, the reaction buffer is a buffer suitable for gel electrophoresis of proteins. Buffers are added in protein preparations to separate proteins from other cell components while preserving the protein structure. This promotes proteins retaining their functions even when the environment around the protein changes. Reaction buffers have a function of maintaining the pH of the protein solution as they prevent changes in pH when an acidic or basic component is added to the protein solution. In some embodiments, the reaction buffer comprises a combination of a Laemmli buffer and mercaptoethanol. The concentration of the Laemmli buffer may be 2× or 4×. In a preferred embodiment, the concentration of the Laemmli buffer is 2×. The addition of β-mercaptoethanol to the buffer promotes the breakage of bigger molecules of proteins that may be formed during the process of extraction of proteins. B-mercaptoethanol breaks disulfide bonds between protein molecules so that bands related to individual polypeptides are formed during electrophoresis. In some embodiments, the β-mercaptoethanol is added to the Laemmli buffer in a volumetric ratio of 10:1 to 25:1 based on a total volume of the reaction buffer. In certain embodiments, the β-mercaptoethanol is added to the Laemmli buffer in a volumetric ratio of 12:1 to 24:1, preferably either 13:1 to 23:1, 14:1 to 22:1, 15:1 to 21:1, 16:1 to 20:1, or more preferably 17:1 to 19:1, and yet more preferably 18:1 to 19:1 based on a total volume of the reaction buffer. In a preferred embodiment, the β-mercaptoethanol is added to the Laemmli buffer in a volumetric ratio of 19:1 based on a total volume of the reaction buffer.

The reaction mixture of protein lysate, comprising at least one target protein, and reaction buffer has a volumetric ratio of 1:1 to 1:20. In one embodiment, the reaction mixture comprises the protein lysate and the reaction buffer in a volumetric ratio of 1:2 to 1:19, preferably either 1:3 to 1:18, 1:4 to 1:17, 1:5 to 1:16, 1:6 to 1:15, 1:7 to 1:14, 1:8 to 1:13, or more preferably 1:9 to 1:12, and yet more preferably 1:10 to 1:11. In a preferred embodiment, the reaction mixture comprises the reaction buffer and the protein lysate comprising at least one target protein in a volumetric ratio of about 1:20.

At step 104, the method 100 includes heating the reaction mixture prepared in step 102. The reaction mixture is heated on a dry bath incubator or a heat block at about 90 to 100° C., preferably either 91 to 99° C., 92 to 98° C., 93 to 97° C., or more preferably 94 to 96° C. In a preferred embodiment, the reaction mixture is heated at about 95° C. The reaction mixture may be heated for 1 to 10 minutes, preferably either 2 to 9 minutes, 3 to 8 minutes, 4 to 7 minutes, or more preferably 5 to 6 minutes, and yet more preferably for about 5 minutes.

At step 106, the method 100 includes loading the reaction mixture in the electrophoresis gel. An electrophoresis chamber is filled with an electrophoresis gel, preferably a polyacrylamide gel. In one embodiment, the gel is a precast gel. In a preferred embodiment, the gel is a mini-protean TGX precast gel. In some embodiments, the gel is made in-house by any known methods in the art. Small wells are created in the gel using a comb to load the reaction mixture and a running buffer. The running buffer facilitates the movement of proteins through the gel during the electrophoresis process. In a preferred embodiment, the running buffer is an SDS-PAGE running buffer. The SDS-PAGE running buffer comprises a mixture of tris, glycine, and sodium dodecyl sulfate (SDS) in water. In some embodiments, the concentration of the SDS-PAGE running buffer is 5×. In a preferred embodiment, the concentration of the SDS-PAGE running buffer is 10 ×. In other embodiments, the running buffer may be any known buffer in the art. For preparing a 10 × running buffer solution, about 30.0 g of tris (tris (hydroxymethyl) aminomethane), about 144.0 g of glycine, and about 10.0 g of SDS are added to 1 liter of water such that the pH of the buffer is about 8.1-8.3. The running buffer is prepared in water, which may be distilled water, bi-distilled water, deionized water, deionized distilled water, reverse osmosis water, tap water, or any other known water of the art. In a preferred embodiment, the water is distilled water. The running buffer comprises a tris/glycine/sodium dodecyl sulfate buffer and water in a volumetric ratio of 1:5 to 1:15 based on a total volume of the running buffer. In certain embodiments, the running buffer comprises the tris/glycine/sodium dodecyl sulfate buffer and water in a volumetric ratio of 1:6 to 1:14, preferably either 1:7 to 1:13, 1:8 to 1:12, or more preferably 1:9 to 1:11, and yet more preferably about 1:9 to 1:10 based on a total volume of the running buffer.

At step 108, the method 100 includes running gel electrophoresis on the electrophoretic gel for a period of 30 to 90 minutes. In some embodiments, the electrophoresis is run for a period of 40 to 80 minutes, preferably either 50 to 70 minutes, or more preferably 55 to 65 minutes, and yet more preferably about 60 minutes. In a preferred embodiment, the electrophoresis is run for a period of about 60 minutes. In some embodiments, the electrophoresis is carried out at 80 to 120 V. In other embodiments, the electrophoresis is carried out at 85 to 115 V, preferably either 90 to 110 V, or more preferably 95 to 105 V, and yet more preferably about 100 V.

At step 110, the method 100 includes rinsing the gel with a transfer buffer. Transfer buffer enables the movement of proteins, separated during electrophoresis, from the gel to a solid support. The transfer buffer can be any standard transfer buffer used in a Western blot. In some embodiments, the transfer buffer is a towbin buffer which is based on tris and glycine.

At step 112, the method 100 includes transferring the blot bands from the gel to a membrane. The transfer of blot bands from the gel may be carried out by a transfer blot turbo transfer system. In some embodiments, the transfer of blot bands from the gel may be done by any methods known in the art. A transfer blot turbo transfer system comprises transfer packs and transfer cassettes. The transfer packs have membranes based on nitrocellulose or polyvinylidene fluoride (PVDF). In one embodiment, the transfer packs with PVDF membranes are used to transfer the blot bands from the gel. In other embodiments, the transfer packs with mini PVDF membranes transfer the blot bands from the gel. The transfer cassettes comprise electrode plates having anions and cations. The blot bands are transferred to the PVDF membrane by a sandwich transfer process wherein gel-containing blot bands are placed between the transfer cassettes and the transfer packs. A predefined protocol can be selected in the turbo transfer system to facilitate the transfer under standard conditions. In some embodiments, the system is run for 20 to 30 minutes, preferably either 21 to 29 minutes, 22 to 28 minutes, 23 to 27 minutes, or more preferably 24 to 26 minutes, and yet more preferably about 25 minutes for transfer of protein bands from the gel to the membrane. In some embodiments, the preferred percentage is at least 50%, 60%, 70%, 80%, or more preferably at least 90%, and more preferably at least 95% of the blot bands from the gel is transferred to the membrane.

At step 114, the method 100 includes contacting the polyvinylidene fluoride membrane with a first blocking buffer. A blocking buffer is used to prevent the non-specific binding of antibodies to a membrane. The blocking buffer also prevents non-specific interactions between the membrane and other reagents that may be used in the detection method. In some embodiments, the first blocking buffer comprises milk powder and saline. The saline is preferably a tris-buffered saline with a non-ionic detergent. In one embodiment, the non-ionic detergent is tween-20, and the saline is a tris-buffered saline with tween-20 (TBST). In another embodiment, the non-ionic detergent may be any detergent known in the art. In a specific embodiment, the first blocking buffer comprises 1× TBST solution and about 2 to 8% weight by volume (w/v, wt/v), preferably 3 to 7% w/v, more preferably 4 to 6% w/v, and yet more preferably about 5% w/v, of milk powder. In a preferred embodiment, the milk powder is a Regilait skimmed milk powder. In some embodiments, the milk powder is any milk powder known in the art. In other embodiments, the blocking binder may be any blocking buffer known in the art.

The PVDF membrane is kept with the first blocking buffer for 20 to 120 minutes. In some embodiments, the PVDF membrane is kept with the first blocking buffer for a period of preferably either 25 to 115 minutes, 30 to 110 minutes, 35 to 105 minutes, 40 to 100 minutes, 45 to 95 minutes, 50 to 90 minutes, 55 to 85 minutes, 60 to 80 minutes, or 65 to 75 minutes. In one preferred embodiment, the PVDF membrane is kept with the first blocking buffer for a period of about 60 minutes. In another preferred embodiment, the PVDF membrane is kept with the first blocking buffer for a period of about 30 minutes. In another preferred embodiment, the PVDF membrane is kept with the first blocking buffer for a period of 30 to 60 minutes.

At step 114, the method 100 further includes contacting the PVDF membrane with the first blocking buffer at temperatures of preferably either −10 to 15° C., −8 to 14° C., −6 to 12° C., −4 to 10° C., −2 to 8° C., 0 to 6° C., or more preferably 2 to 4° C., and yet more preferably about 4° C. A preferred temperature can be obtained by keeping the PVDF membrane with the first blocking buffer in a refrigerator.

At step 116, the method 100 includes contacting the PVDF membrane with a second 10 blocking buffer. The second blocking buffer comprises an antibody cocktail. The antibody cocktail is a single reagent and comprises a mixture of primary and secondary antibodies. The primary antibody is specific for the epitope present on the target protein. Accordingly, when the target protein is selected from a group consisting of β-actin, GAPDH, p62, AMPK, CHOP, JNK, and TAP-1, the primary antibody is selected from a group consisting of mouse monoclonal antibody to β-actin, rabbit polyclonal antibody to GAPDH, rabbit monoclonal antibody to p62, rabbit polyclonal antibody to AMPK, mouse monoclonal antibody to CHOP, mouse monoclonal antibody to JNK, anti-TAP-1 antibody, and mouse monoclonal antibody to lamin B1. In some embodiments, the target protein may be any protein known in the art. In some embodiments, the primary antibody may be any antibody known in the art. The primary antibody specifically binds the target protein.

The secondary antibody is selected for its specificity for binding sites on the primary antibody. The secondary antibodies help detect target antigens by binding to the primary antibodies bound to the target antigens. In an exemplary embodiment, the secondary antibody in the antibody cocktail is selected from a group consisting of goat anti-mouse HRP conjugate and goat anti-rabbit HRP conjugate. In another embodiment, the secondary antibody may be any antibody known in the art.

In one embodiment, the second blocking buffer comprises a primary and a secondary antibody in a ratio of 0.1:1 to 10:1. In other embodiments, the second blocking buffer comprises a primary and a secondary antibody in a ratio of preferably either 0.2:1 to 9:1, 0.3:1 to 8:1, 0.4:1 to 7:1, 0.5:1 to 6:1, 0.6:1 to 5:1, 0.7:1 to 4:1, 0.8:1 to 3:1, 0.9:1 to 2:1, or about 1:1.

In some embodiments, the antibodies are diluted with blocking buffers to obtain various concentrations of antibodies. The dilutions are prepared by mixing primary and secondary antibodies with the first blocking buffer to obtain final working dilutions of both antibody types. In certain embodiments, the primary antibody has a final working dilution of 1:800 to 1:1200. In other embodiments, the primary antibody has a final working dilution of preferably either 1:850 to 1150, 1:900 to 1:1100, or more preferably 1:950 to 1:1050, and yet more preferably about 1:1000. In an exemplary embodiment, the primary antibody has a final working dilution of 1:1000. In an embodiment, the secondary antibody has a final working dilution of 1:2500 to 1:3500. In some other embodiments, the secondary antibody has a final working dilution of preferably either 1:2600 to 1:3400, 1:2700 to 1:3300, 1:2800 to 1:2300, or more preferably 1:2900 to 1:3100, and yet more preferably about 1:3000. In an exemplary embodiment, the secondary antibody has a final working dilution of 1:3000. The final working dilution may be based on a total volume of the blocking buffers and primary and secondary antibodies.

In some embodiments, the PVDF membrane is contacted with the second blocking buffer for a period of 40 to 80 minutes. In other embodiments, the PVDF membrane is contacted with the second blocking buffer for a period of preferably either 42 to 78 minutes, 45 to 75 minutes, 50 to 70 minutes, or more preferably 55 to 65 minutes, and yet more preferably about 60 minutes.

In some embodiments, the PVDF membrane is incubated with the second blocking buffer at temperatures of preferably either −10 to 15° C., −8 to 14° C., −6 to 12° C., −4 to 10 ° C., −2 to 8° C., 0 to 6° C., or 2 to 4° C. A preferred option is to incubate the PVDF membrane with the second blocking buffer in a refrigerator.

In some embodiments, the secondary antibody is conjugated to the primary antibody before conjugating the primary antibody to the target protein. For example, at least one primary antibody may be conjugated to the target protein, but at least one secondary antibody may be conjugated to the at least one primary antibody before the at least one primary antibody may be conjugated to the target protein.

In some embodiments, the secondary antibody is conjugated to a signal generator that generates a signal in response to a chemical reaction indicating the presence of a target protein. The signal generator can be an enzyme or some fluorescent compound. In some embodiments, the signal generator may be any signal generator known in the art. The secondary antibodies may be conjugated to a fluorescent compound, which generates a fluorescent light that can be detected by a fluorescent microscope. The fluorescent compounds may include dyes such as Fluorescein Isothiocyanate, rhodamine, cyanine, aminomethylcoumarin acetate (AMCA), and the like. Alternatively, the secondary antibodies may be conjugated to an enzyme wherein the oxidation of a chemical compound in the presence of the enzyme produces a visible signal that can be detected by a suitable imaging system. In a specific embodiment, the secondary antibodies are conjugated to an enzyme wherein the enzyme can be an alkaline phosphatase (AP), a horse radish peroxidase (HRP), and the like. In a preferred embodiment, the secondary antibodies are conjugated to a horse radish peroxidase.

At step 118, the method 100 includes washing the PVDF membrane with a wash buffer. Washing of the membrane is done to remove any substance, including the reagents and antibodies, that are non-specifically bound to the membrane. Suitable wash buffers that can be used for washing the membrane include tris-buffered saline, phosphate-buffered saline mixed with a detergent, or any known wash buffer in the art. For example, tris-buffered saline with tween 20 (TBST) or phosphate-buffered saline with tween 20 (PBST) can be used as wash buffers. In a preferred embodiment, TBST at a concentration of 1× may be used to wash the PVDF membrane. In some embodiments, the membrane is washed by incubating the membrane with the wash buffer and simultaneously agitating the membrane for faster removal of unwanted substances. The agitation may be carried out by placing the membrane in the buffer on a rotator, a shaker, or other suitable agitator. The membrane may be washed 1 to 3 times for a period of 4 to 8 minutes. In one embodiment, the membrane and the wash buffer are placed on a rotator for a period of 4 to 7 minutes, preferably 5 to 6 minutes. In a specific embodiment, the membrane is washed at least 3 times for a period of 5 minutes each on a rotator.

At step 120, the method 100 includes addition of detection reagents to the washed PVDF membrane. The PVDF membrane is incubated with suitable detection reagents, wherein a mixture of detection reagents may be used. In one embodiment, method 100 includes adding a clarity Western substrate ECL kit to the PVDF membrane. The kit comprises a mixture of a peroxide solution and a luminol enhancer solution in equal proportions. In certain embodiments, the peroxide solution is a clarity Western peroxide reagent, and the luminol enhancer solution is a clarity Western luminol/enhancer reagent and both reagents are mixed in a 1:1 proportion prior to their incubation with the membrane. The PVDF membrane is incubated with the detection reagents for a period of preferably either 30 to 90 seconds, 35 to 85 seconds, 40 to 80 seconds, 45 to 75 seconds, 50 to 70 seconds, or more preferably 55 to 65 seconds, and yet more preferably about 60 seconds.

At step 122, the method 100 includes imaging the PVDF membrane to detect the target protein. The luminol in the clarity Western enhancer reagent is oxidized in the presence of peroxide. The oxidation takes place by the action of the enzyme horse radish peroxidase (HRP), which is conjugated to the secondary antibodies. The enzymatic action results in the formation of 3-aminophthalate dianion and generation of a signal in form of light. The light is visualized using an imaging system, indicating the presence of the target protein.

In some embodiments, a method of forming a protein lysate is described. The method includes removing cells comprising a target protein from a cell culture and mixing the cells with a lysing and extraction buffer. The lysing and extraction buffers promote lysing of cells and extraction of proteins from the lysed cells. The lysing and extraction buffers may be the same or different. Suitable examples of lysing and extraction buffers include NP-40, RIPA, SDS (sodium dodecyl sulfate) lysis buffer, and ammonium chloride potassium lysing buffer, and the like. In a preferred embodiment, a RIPA buffer is used for cell lysis and extraction of proteins. The cells comprising the target proteins are mixed with RIPA buffer to form a first mixture.

In some embodiments, at least one protease and phosphatase inhibitor are added to the first mixture. The protease and phosphatase inhibitors prevent the degradation of extracted proteins and preserve the phosphorylated protein residues. Suitable protease inhibitors include aprotinin, leupeptin, pepstatin A, bestatin, EDTA disodium salt, pepstain A, and the like. Suitable phosphatase inhibitors include okadaic acid, microcystin-LR, tautomycin, β-glycerophosphate, sodium fluoride, imidazole, sodium molybdate, and the like. One or more protease and phosphatase inhibitors may be added to the first mixture.

The first mixture having protease and phosphatase inhibitors is kept on ice for a period of preferably either 20 to 30 minutes, 21 to 29 minutes, 22 to 28 minutes, 23 to 27 minutes, or more preferably 24 to 26 minutes, and yet more preferably about 25 minutes.

The incubation of the first mixture with inhibitors is followed by centrifugation in a suitable centrifuge. In certain embodiments, the first mixture with inhibitors is centrifuged in a microcentrifuge at a speed of about preferably either 10,000 to 15,000 rpm, 11,000 to 14,000 rpm, or more preferably 12,000 to 13,000 rpm, and yet more preferably about 13,500 rpm. In some embodiments, the centrifuging occurs for a period of 1 to 10 minutes, preferably 2 to 9 minutes, 3 to 8 minutes, 4 to 7 minutes, or more preferably 5 to 6 minutes, and yet more preferably about 5 minutes to separate the protein lysate from the mixture. The supernatant from centrifuged mixture is decanted to collect the protein lysate which may be stored at temperatures of about −15 to −25° C., preferably about −20° C.

The present disclosure also provides antibody cocktails that have good stability at low temperatures. The antibody cocktails comprise both primary and secondary antibodies which can be preserved for at least a period of one year when stored at temperatures of about −30 to −10° C., preferably −25 to −15° C., or about −20° C. Examples of antibody cocktails include a combination of mouse monoclonal antibody to beta actin and goat anti-mouse HRP conjugated secondary antibody; a combination of rabbit polyclonal antibody to GAPDH and goat anti-rabbit HRP conjugated secondary antibody; a combination of rabbit polyclonal antibody to GAPDH, rabbit monoclonal antibody to p62, and goat anti-rabbit HRP conjugated secondary antibody; a combination of mouse monoclonal antibody to beta actin, rabbit polyclonal antibody to GAPDH, rabbit monoclonal antibody to p62, goat anti-mouse HRP conjugated secondary antibody, and goat anti-rabbit HRP conjugated secondary antibody; a combination of mouse monoclonal antibodies to JNK and CHOP, rabbit polyclonal antibody to AMPK, goat anti-mouse HRP conjugated secondary antibody, and goat anti-rabbit-HRP conjugated secondary antibody; and, a combination of mouse monoclonal antibody to TAP-1 and goat anti-mouse HRP conjugated secondary antibody.

### EXAMPLES

The disclosure will now be illustrated with working examples intended to demonstrate the working of the disclosure and not to restrictively imply any limitations on the scope of the present disclosure. The working examples depict an example method of the present disclosure.

The following examples demonstrate the preparation of protein lysate and a method for immunodetection as described herein. The examples are provided solely for illustration and are not to be construed as limitations of the present disclosure, as many variations thereof are possible without departing from the spirit and scope of the present disclosure.

### Example 1: Materials and Methods

A master mix was prepared using RPMI-1640 medium supplemented with 10% Fetal Bovine Serum (FBS), 1% penicillin-streptomycin (pen/strep), and 1% L-glutamine (Glutamax reagent). Cells were seeded from the initial vial containing 1×10⁶ cells onto T25 flasks and grown to ˜80% confluency for all experiments. Cell lines were cultured at 37° C. in a 5% CO₂ atmosphere incubator.

Passaging cell lines in culture was performed once they reached ˜80% confluency. Flasks were washed twice using PBS, removing excess FBS, as the presence of FBS would hinder the trypsinization process. 3 mL of Trypsin-EDTA (1×) was added. Culture flasks were incubated for 3 minutes at 37° C. then properly shaken to ensure all cells detached from the flasks. 5 mL of media was added to stop further trypsinization that may degrade the cells. Further mixing was performed using a pipette to help cells completely detach, and all media was transferred to a 15 mL falcon tube to be centrifuged at 1500 rpm for 5 minutes. After centrifugation, the supernatant was completely discarded and 1 mL of prepared RPMI-1640 media was added and mixed well with the pellet. 5 mL of media was added later and properly mixed. The contents of the falcon tube were mixed and divided equally and placed into newly labeled T25 flasks containing 10 mL of RPMI-1640 media. These flasks were placed in a 5% CO₂ atmosphere incubator at 37° C. A change of media was performed when needed until cells reached ˜80% confluency, cells were split into T25 flasks, and the passage process was repeated to collect proper protein amounts for further testing.

### Example 2: Protein Extraction

Protein extraction was performed using radio-immunoprecipitation assay (RIPA) lysing and extraction buffer. Cell lysis has been shown to disrupt cellular components, creating an upregulation of endogenous enzymes that may affect result outcomes by increasing degradation. Protease and phosphatase inhibitors were added to prevent the degradation of extracted proteins and preserve phosphorylated residues during cell disruption.

Media was removed from the T25 cell culture and washed twice with ice-cold PBS. All reagents were placed on an ice tray. 1 mL of RIPA buffer was added to the flask with 10 μL of both phosphatase and protease inhibitors (after a proper mix of the bottles). The flasks were properly mixed and placed on ice for 30 minutes with the reagents evenly distributed in the flask. After the end of the 30-minute incubation period, flasks were scraped using a cell scraper to remove all attached cells. The flasks were examined under the microscope, making sure no cells were still attached. Afterwards, the mixture was transferred into labeled Eppendorf tubes, vortexed, and centrifuged in a microcentrifuge for 5 minutes at 13,500 rpm. The supernatant was then transferred into newly labeled Eppendorf tubes and stored at −20° C.

### Example 3: Protein Quantification

Bicinchoninic acid (BCA) assay was performed, following Thermofisher protocol, on extracted protein lysates to determine the total protein concentration for each sample. Quantification of protein concentrations is used to unify protein amounts for later Western blotting experiments. Protein lysates were thawed on ice and then diluted with phosphate-buffered saline (PBS) in a 1:10 ratio. 80 μL was prepared for each sample by diluting 8 μL of the sample with 72 μL of PBS. The BCA solution was prepared by mixing reagent A with reagent B in a 50:1 ratio. 12.5 mL of reagent A (sodium carbonate, sodium bicarbonate, bicinchoninic acid (BCA), sodium tartrate) was mixed with 0.25 ml of reagent B (copper(II) sulfate (CuSO₄)). In a microtiter plate (96 well plates) placed on ice, 20 μL of blank (PBS), 7 protein standards, and sample lysates were added to pre-defined wells in triplicate. 200 μL of prepared BCA solution was added to all the wells. The plate was then placed on a rotator to be thoroughly mixed for 30 seconds. The microtiter plate was covered in foil, as it is light sensitive, and incubated at 37° C. for 30 minutes. The microtiter plate was then removed from the incubator and placed onto the absorbance plate reader (BioTek, ref. ELX808) to measure the absorbance near 420 nm. The average of the triplicate samples was calculated by the automated plate reader and then analyzed against the standard curve obtained using Microsoft Excel software.

### Example 4: Protein Analysis

Sample preparation: Protein lysate samples were prepared based on protein concentrations calculated from the BCA assay. Aliquots of protein corresponding to known amounts of total protein of each cell line were obtained and diluted with calculated amounts of RNAse-free water in Eppendorf tubes. All sample concentrations were unified in a total of 50 μL of sample and water mixture. Sample buffer was prepared by mixing 475 μL of 2× Laemmli buffer (BioRad, cat: 161-0737) with 25 μL of β-mercaptoethanol (Plusone, Code no. 17-1317-01), and 50 μL of sample buffer was added to each sample. Eppendorf tubes containing the sample mixture were sealed with parafilm, vortexed, and placed on a heat block for 5 minutes at 95° C.

Gel electrophoresis: An electrophoresis chamber was filled with SDS-PAGE running buffer that was prepared by adding 100 mL 10 × tris-glycine-SDS-PAGE (TGS) running buffer to 900 mL deionized water. 2 mini-protean TGX precast gel (4-15%, 10-well comb, 50 L/well) cassettes were placed inside the chamber (after removing comb and tape). SDS-PAGE buffer was poured to fill the inner chamber and reach the top of the gels. 50 μL of samples were then loaded on the gel starting from the second well, leaving the first well for the 10 μL protein standard ladder (Bio-Rad, Cat. No. 161-0363). Electrodes were then connected for the gel to run at 100 V for approximately 60 minutes, separating proteins based on their molecular weight.

### Western Blot Technique (Conventional vs. Non-Conventional (the Proposed Improved Protocol)

Conventional Method: Once the electrophoretic run was completed, cassettes were opened for gel retrieval. Gels were rinsed with the transfer buffer. Transfer-Blot Turbo Transfer System (serial no. 690BR013881) was used to blot bands from the gel onto the polyvinylidene fluoride (PVDF) membrane (Trans-blot turbo transfer pack, Cat. No. 1704156). Mini PVDF transfer packs were used and assembled via the sandwich method in the transfer cassette for each gel. The transfer was at standard settings for 25 minutes. A blocking buffer was prepared by adding 1× TBST to dissolve 5% w/v of dried milk powder (Regilait skimmed milk). PVDF membranes were blocked for 30 minutes to 1 hour in the fridge, ensuring full membrane coverage.

Primary antibodies were then diluted with blocking buffer at pre-defined concentrations, as described in Tables 1 and 2. Membranes were then incubated with primary antibodies overnight at 4° C. Membranes were washed 3 times in 1× TBST for 5 minutes each on a rotator. Then, the membranes were stained with HRP-linked secondary goat anti-mouse/rabbit diluted in blocking buffer for 1 hour in the fridge, ensuring full coverage of the membrane. The membrane was washed 3 times in 1× TBST for 5 minutes each on a rotator. A 1:1 ratio of ECL Western blot detection reagents (BioRad, cat: 170-5061) were mixed, used on the membrane, and incubated for 1 minutes. BIO-RAD ChemiDoc MP imaging system (serial no. 731BR02984) was used to develop the membrane on chemiluminescent blot standard settings. Band analysis was performed using Image J software. The housekeeping protein was detected using β-actin (a mouse anti-β-actin monoclonal antibody). Experiments were carried out in triplicate on 3 separate occasions for each ovarian cancer cell line or as indicated otherwise.

Non-conventional method (the proposed improved protocol): Once the electrophoretic run was completed, cassettes were opened for gel retrieval. Gels were rinsed with the transfer buffer. Transfer-Blot Turbo Transfer System (serial no. 690BR013881) was used to blot bands from the gel onto the PVDF membrane (Bio-Rad, Trans-blot turbo transfer pack, Cat. No. 1704156). Mini PVDF transfer packs were used and assembled via the sandwich method in the transfer cassette for each gel. The transfer was at standard settings for 25 minutes.

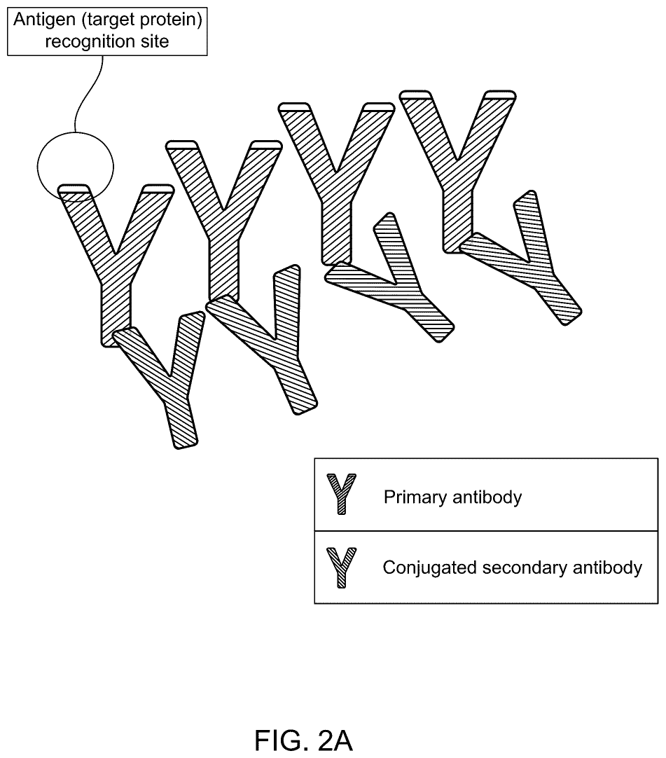

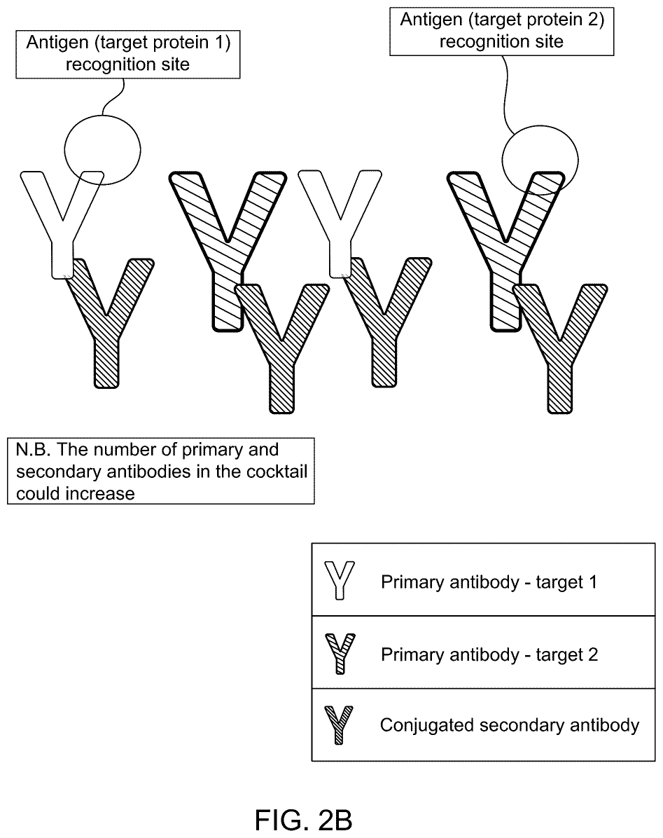

Alternatively, probed membranes could be stripped and re-probed with the proposed antibodies cocktails, indicating that these cocktails work in different experimental settings of Western blotting (blots of stripped membranes are highlighted in the results section). Blocking buffer was prepared by adding 1× TBST to dissolve 5% w/v of dried milk powder (Regilait skimmed milk). PVDF membranes were blocked for 30 minutes to 1 hour in the fridge, ensuring full coverage of the membrane. FIG. 2A and 2B depict the proposed antibody cocktails that could contain a primary and a secondary antibody (FIG. 2A) or several primary antibodies and a secondary antibody for multiplex detection (FIG. 2B).

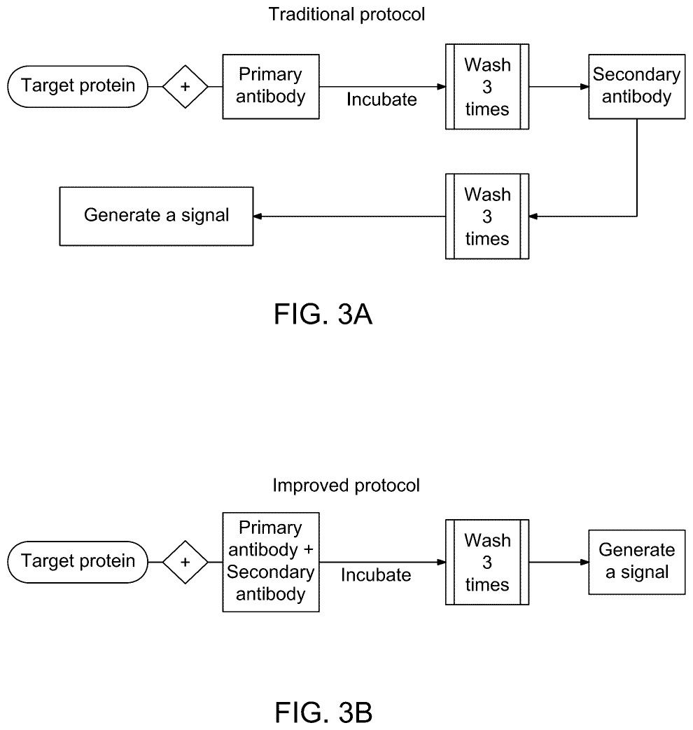

Primary and secondary antibodies were diluted together with blocking buffer at determined concentrations (antibodies used are summarized in Tables 1 and 2), and membranes were covered with antibody cocktails and incubated in the fridge for 1 hour. The membrane was washed 3 times in 1× TBST for 5 minutes each on a rotator. A 1:1 ratio of ECL Western blot detection reagents (BioRad, cat: 170-5061) were mixed, used on the membrane, and incubated for 1 minute. BIO-RAD ChemiDoc MP imaging system (serial no. 731BR02984) was used to develop the membrane on chemiluminescent blot standard settings. Experiments were carried out on ovarian and colon cancer cell lines. FIG. 3A illustrates a traditional protocol for immunodetection. FIG. 3B illustrates an improved protocol (method of the present disclosure) for immunodetection.

The stability of the proposed antibody cocktails was checked after being stored at −20° C. for approximately 1 year (Table 2). This was done after an initial experiment in which a premade mixture of “stock” primary and secondary antibodies was stored at 4° C. for 72 hours before being diluted and used to probe β-actin (bottom of Table 1). This denotes the possibility of manufacturing 5 these cocktails for commercial purposes.

TABLE 2
Antibodies cocktails used for stability
checks of pre-made antibodies mixtures.

| | Primary antibody | Secondary antibody |
|---|---|---|
| Cocktail 1 | Mouse monoclonal | Goat anti-mouse HRP |
| Co-detection of | antibody to β-actin | conjugate (Bio-Rad, |
| β-actin and GAPDH | (Abcam, Cat. No. | Cat. No. 170-5047) |
| in OVCAR4 ovarian | ab8226) | |
| cancer cell lines | Rabbit polyclonal | Goat anti-rabbit HRP |
| | antibody to GAPDH | conjugate (Bio-Rad, |
| | (Molequle-On, Cat. | Cat. No. 170-6515) |
| | No. KT1002) | |
| Cocktail 2 | Rabbit polyclonal | Goat anti-rabbit HRP |
| Co-detection of | antibody to GAPDH | conjugate (Bio-Rad, |
| GAPDH and p62 in | (Molequle-On, Cat. | Cat. No. 170-6515) |
| HCT-116 colon | No. KT1002) | |
| cancer cell lines | Rabbit monoclonal | |
| | antibody to p62 | |
| | (SQSTM1) (Abcam, | |
| | Cat. No. ab109012) | |
| Cocktail 3 | Mouse monoclonal | Goat anti-mouse HRP |
| Co-detection of β- | antibody to β-actin | conjugate (Bio-Rad, |
| actin, GAPDH, and | (Abcam, Cat. No. | Cat. No. 170-5047) |
| p62 in HCT-116 colon | ab8226) | |
| cancer cell lines | Rabbit polyclonal | Goat anti-rabbit HRP |
| | antibody to GAPDH | conjugate (Bio-Rad, |
| | (Molequle-On, Cat. | Cat. No. 170-6515) |
| | No. KT1002) | |
| | Rabbit monoclonal | |
| | antibody to p62 | |
| | (SQSTM1) (Abcam, | |
| | Cat. No. ab109012) | |
| Cocktail 4 | Rabbit polyclonal | Goat anti-rabbit HRP |
| Co-detection of | antibody to AMPK | conjugate (Bio-Rad, |
| AMPK, CHOP, and | alpha 1 (Proteintech ®, | Cat. No. 170-6515) |
| JNK in OVCAR | Cat. No. 10929-2AP) | |
| 4 cancer cells | Mouse monoclonal | Goat anti-mouse HRP |
| | antibody to JNK | conjugate (Bio-Rad, |
| | (Proteintech ®, Cat. | Cat. No. 170-5047) |
| | No. 66210-1-Ig) | |
| | Mouse monoclonal | |
| | antibody to CHOP | |
| | (GADD153) | |
| | (Proteintech ®, Cat. | |
| | No. 60304-1-Ig) | |
| Cocktail 5 | Mouse monoclonal | Goat anti-mouse HRP |
| detection of TAP- | antibody to TAP-1 | conjugate (Bio-Rad, |
| 1 in PEO6 ovarian | (Molequle-On, Cat. | Cat. No. 170-5047) |
| cancer cells | No. AB-M-067) | |

Primary antibodies final working dilution = 1:1000
Secondary antibodies final working dilution = 1:3000

### Results

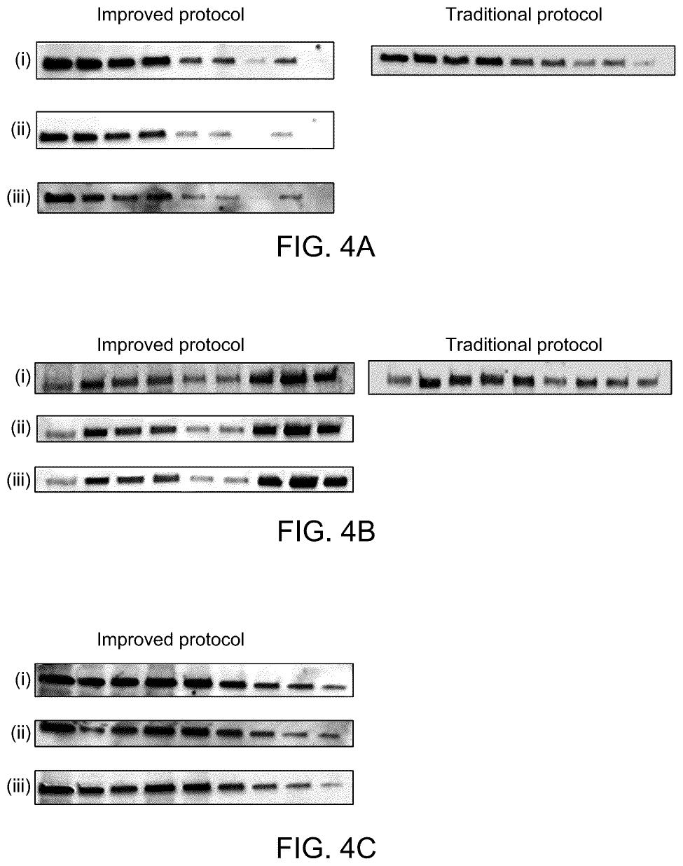

Western blot analysis is used to evaluate the level of protein expression on cell lines that may be used to explain abundance, modifications, and/or interactions of certain proteins, which may help in the diagnosis of certain diseases, one of which is cancer, which may also lead to appropriate treatment regimens. The protein band densities were analyzed using two different Western blot protocols; one was the conventional method and the other was the non-conventional method proposed herein to improve the traditionally used protocol. The proposed method herein detects a single protein using a simultaneous incubation with primary and secondary antibody cocktails using both protocols. The results showed approximately similar band densities (analysis was done for β-actin), which is interpreted as no statistical differences were obtained between the two methods (the conventional vs. non-conventional), as shown in FIG. 4A and FIG. 4B. The detection of β-actin, glyceraldehyde 3-phosphate dehydrogenase (GAPDH), and transporter associated with antigen processing 1 (TAP-1) proteins in a series of ovarian cancer cell lines is performed by improved and traditional Western blotting protocols. Blots shown in FIG. 4A-FIG. 4C depict three different proteins detected using the Western blotting technique in different cell lysates. The band densities of β-actin and TAPI obtained by the improved protocols are comparable to those depicted by the traditional protocol. GAPDH was detected using the improved Western blotting protocol with clear band intensities, as with the other two proteins. The results confirm that co-incubation of the membrane with the primary and secondary antibodies simultaneously is a successful approach, which does not affect signal generation and antibody-antigen interaction.

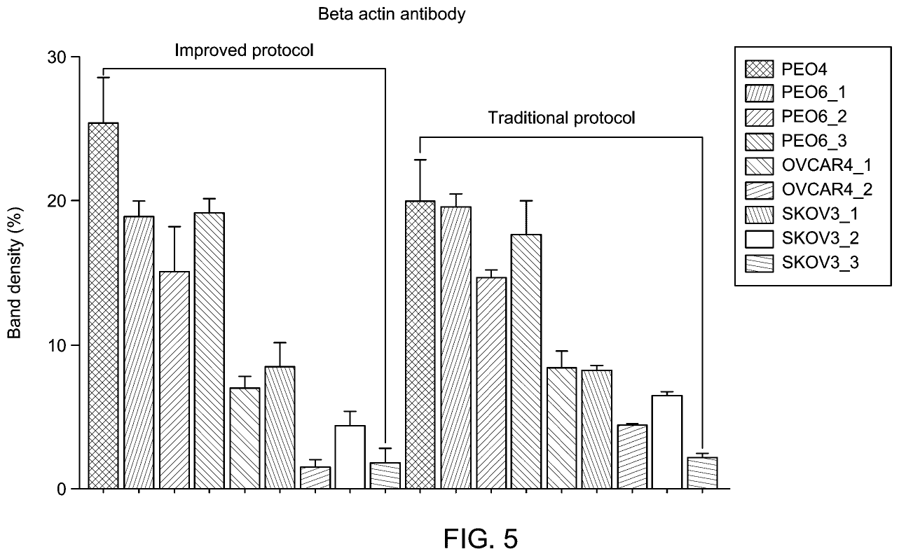

β-actin and TAP1 were detected using the traditional procedure and the improved protocol of the present disclosure, as seen in FIG. 4A and FIG. 4B, respectively. Also, GAPDH was detected in the same cell lysates using the improved protocol (FIG. 4C). The band densities were analyzed using ImageJ software, and the comparison between band densities from traditional and improved protocols is shown in FIG. 5 for the β-actin antibody. Analysis of variance on ranks was employed to compare the mean ranks of each pair in the improved and the traditional protocol. There was no significant difference between the mean band densities obtained for 3 different blots by the improved or the traditional protocols. Band densities are the mean values of 3 different blots. The numbers (_1, _2, _3) in the FIG. 5 legend indicate different cell passages.

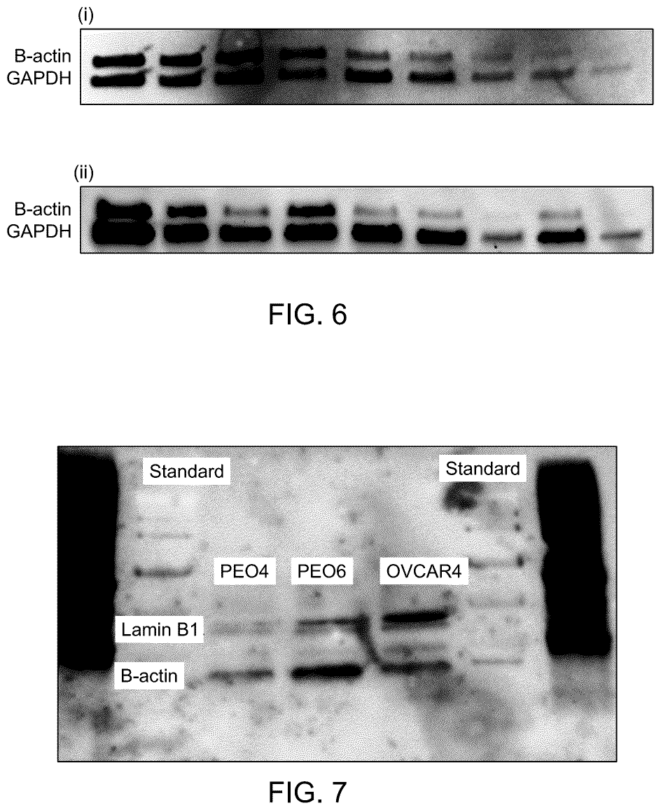

In addition, multiplex Western blotting was used to detect multiple target proteins on the same membrane with a single incubation step and a single detection step. The results, shown in FIG. 6 and FIG. 7, indicated the possibility of detecting β-actin and GAPDH and β-actin and lamin B1 proteins simultaneously, which means that a single incubation with two different primary antibodies and a secondary antibody simultaneously is a successful approach. This diminishes the need to repeat the procedure to detect each target separately and allows co-detection of the target and the internal control on the same membrane simultaneously.

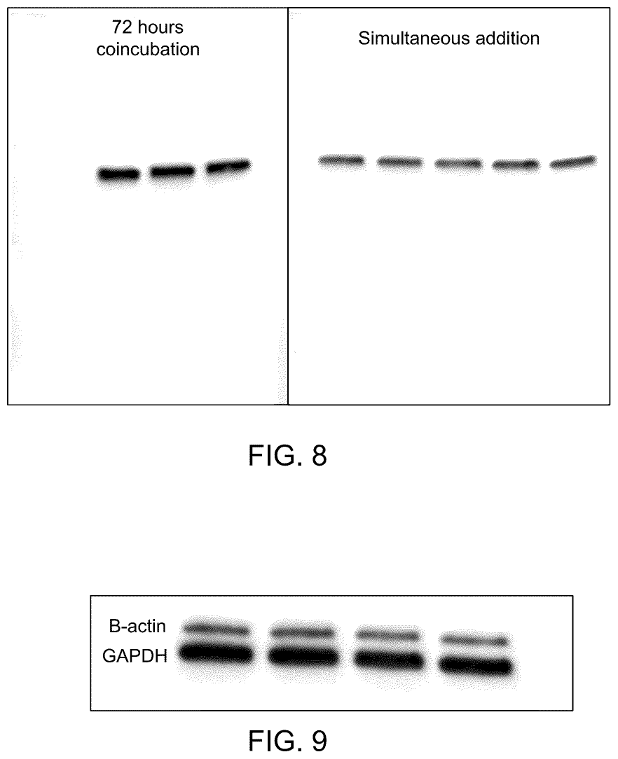

Moreover, FIG. 8 shows a representative experiment indicating the potential to use a premade antibody cocktail for immunodetection using the Western blotting technique. This illustrates the possibility of commercial preparation of such cocktails. Additionally, the potential multiplexing of the improved protocol for concomitant detection of multiple proteins was investigated. The results showed that β-actin and GAPDH were co-detected on the same blot in the same ovarian cancer cells using the antibodies cocktails that were listed in Table 1 (FIG. 6). In a similar context, β-actin and lamin B1 were co-detected in PEO4, PEO6, and OVCAR4 using the suggested protocol (FIG. 7).

Stability Test: To assess the stability of the proposed antibodies cocktails, mixtures stored at −20° C. for 1 year were used to probe target proteins. Western blotting procedure or membrane stripping was performed as described previously. The blots depict the results of the detection of single and multiple proteins.

FIG. 8 shows the detection of β-actin using a pre-made cocktail of stock primary and secondary antibodies that were stored in the fridge for 72 hours before being diluted, as indicated above, and applied on the membrane to probe β-actin protein in PEO6 cells. The results were compared with the previously explained method (improved protocol) involving simultaneously adding primary and secondary antibodies together for detection without making a premade mixture. Both signals were comparable, supporting the proposed cocktails' stability and usability.

Co-detection of β-actin and GAPDH was repeated in OVCAR4 cells using an antibody cocktail stored at −20° C. for 1 year. The results are shown in FIG. 9. The antibody cocktail included a mouse monoclonal antibody to β-actin, goat anti-mouse-HRP conjugated secondary antibody, rabbit polyclonal antibody to GAPDH, and goat anti-rabbit-HRP conjugated secondary antibody. The results support the stability of the antibody cocktail for 1 year at −20° C.

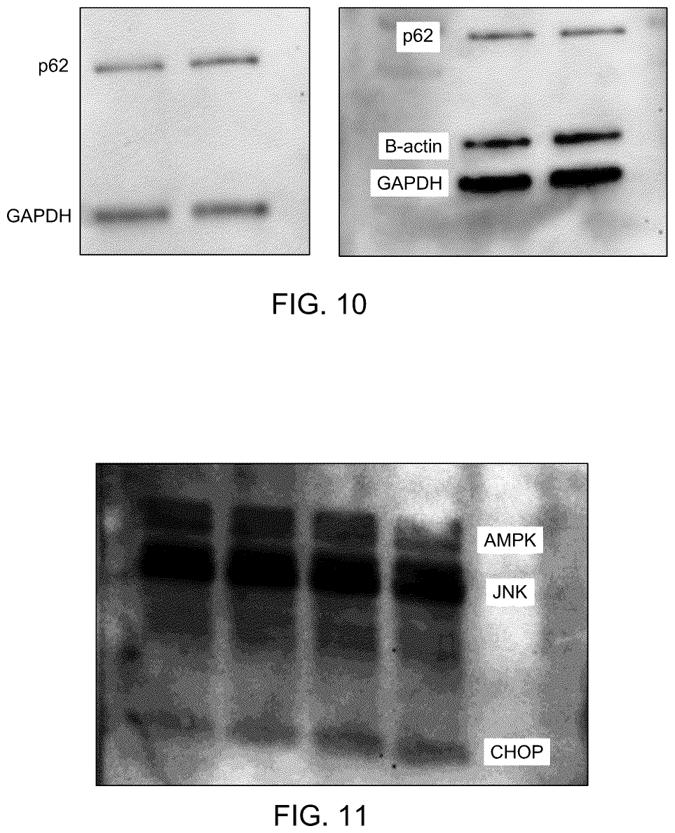

In addition, β-actin, GAPDH, and p62 (SQSTM1) were co-detected in HCT-116 colon cancer cells using an antibody mixture stored at −20° C. for 1 year. This result indicates the potential to increase the number of antibodies in the proposed antibodies cocktails to detect many target proteins simultaneously. Likewise, GAPDH and p62 were co-detected in HCT-116 colon cancer cells using an antibodies cocktail that was stored for 1 year at −20° C. These results for the detection of proteins in HCT-116 cells are shown in FIG. 10. For detection of GAPDH and p62 (left), the antibody cocktail consists of a rabbit polyclonal antibody to GAPDH and a rabbit monoclonal antibody p62 and a goat anti-rabbit HRP conjugated antibody. For co-detection of β-actin, GAPDH, and p62 (right), the antibody mixture contains a mouse monoclonal antibody to beta actin, a rabbit polyclonal antibody to GAPDH a rabbit monoclonal antibody to p62, a goat anti-mouse-HRP conjugated secondary antibody and a goat anti-rabbit-HRP conjugated secondary antibody. The antibody cocktail was stored at −20° C. for one year.

Another antibody combination was used to co-detect AMPK (5′ adenosine monophosphate-activated protein kinase), JNK (c-Jun N-terminal kinase), and CHOP (C/EBP homologous) proteins in OVCAR4 ovarian cancer cells. The antibody mixture was stored at −20° C. for one year before being used to probe the mentioned proteins. For co-detection of AMPK, CHOP, and JNK, the antibody mixture contains mouse monoclonal antibodies to JNK and CHOP, a rabbit polyclonal antibody to AMPK, a goat anti-mouse-HRP conjugated secondary antibody and a goat anti-rabbit-HRP conjugated secondary antibody. The antibody cocktail was stored at −20° C. for one year. The results in FIG. 11 depict the blot of detection.

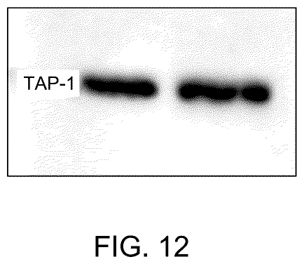

TAP-1 protein was also detected in PEO6 ovarian cancer cells. The antibody cocktail comprised a mouse monoclonal antibody to TAP-1 and a goat anti-mouse HRP conjugated secondary antibody. The antibody cocktail used to probe TAP-1 in PEO6 cells (FIG. 12) was stored at −20° C. for one year and used to detect TAP-1 in PEO6 cells. The antibody cocktails used are listed in Table 2.

These results indicate that primary and secondary antibody cocktails can be made, stored, and used in Western blotting techniques either for singular or multiplex detection purposes. The results also showed the possibility of commercializing these cocktails based on the stability checks for the investigated antibodies. The results also indicate the possibility of making cocktails from multiple antibodies which enable the co-detection of a complete pathway component in a single run.

Numerous modifications and variations of the present disclosure are possible in light of the above teachings. It is therefore to be understood that within the scope of the appended claims, the disclosure may be practiced otherwise than as specifically described herein.

1. A method of immunodetection, comprising:
mixing a protein lysate comprising a target protein and a reaction buffer to form a reaction mixture;
heating the reaction mixture for 1 to 10 minutes at a temperature of 90 to 100° C.;
loading the reaction mixture in a well of a gel in an electrophoresis chamber,
wherein the electrophoresis chamber comprises an SDS-PAGE running buffer,
running gel electrophoresis on the gel at 80 to 120 V for 30 to 90 minutes;
rinsing the gel with a transfer buffer;
transferring blot bands from the gel to a polyvinylidene fluoride membrane;
contacting the polyvinylidene fluoride membrane with a first blocking buffer for 20 to 120 minutes at a temperature of −10 to 15° C.;
contacting the polyvinylidene fluoride membrane with a second blocking buffer for 40 to 80 minutes at a temperature of −10 to 15° C.,
wherein the second blocking buffer comprises an antibody cocktail,
wherein the antibody cocktail comprises a primary antibody and a secondary antibody,
wherein the primary antibody is selected from a group consisting of mouse monoclonal antibody to beta actin, anti-TAP-1 antibody, rabbit polyclonal antibody to GAPDH, and mouse monoclonal antibody to lamin B1,
wherein the secondary antibody is selected from a group consisting of goat anti-mouse HRP conjugate and goat anti-rabbit HRP conjugate,
washing the polyvinylidene fluoride membrane with a wash buffer;
incubating the polyvinylidene fluoride membrane with a mixture of detection reagents for 30 to 90 seconds; and
imaging the polyvinylidene fluoride membrane to detect the target protein.

2. The method of claim 1, wherein the second blocking buffer comprises a primary antibody and a secondary antibody in a ratio of 0.1:1 to 10:1.

3. The method of claim 1, wherein the reaction buffer comprises a 2× Laemmli buffer and β-mercaptoethanol in a volumetric ratio of 10:1 to 25:1.

4. The method of claim 1, wherein a volumetric ratio of the protein lysate to the reaction buffer is from 1:1 to 1:20.

5. The method of claim 1, wherein the gel is a mini-protean TGX precast gel.

6. The method of claim 1, wherein the SDS-PAGE running buffer comprises a 10 × tris/glycine/sodium dodecyl sulfate buffer and water in a volumetric ratio of 1:5 to 1:15.

7. The method of claim 1, wherein the transferring occurs using a sandwich transfer process with a transfer-blot turbo transfer system for 20 to 30 minutes.

8. The method of claim 1, the first blocking buffer comprises a 2 to 8% wt/v milk powder and 1× TBST solution.

9. The method of claim 1, wherein the primary antibody has a final working dilution of 1:800 to 1:1200.

10. The method of claim 1, wherein the secondary antibody has a final working dilution of 1:2500 to 1:3500.

11. The method of claim 1, wherein the mixture of detection reagents is a Clarity Western peroxide reagent and a clarity Western luminol/enhancer reagent.

12. The method of claim 1, wherein the target protein at least one selected from a group consisting of β-actin, GAPDH, p62, AMPK, CHOP, JNK, and TAP-1.

13. The method of claim 1, wherein the target protein is detected in cells in a group consisting of OVCAR4, PEO4, PEO6, SKOV3, and HCT-116.

14. The method of claim 1, wherein the antibody cocktail is stable for at least one year at a temperature of −30 to −10° C.

15. The method of claim 1, further comprising:
removing cells comprising the target protein from a culture;
mixing the cells comprising the target protein with a RIPA lysing and extraction buffer to form a first mixture;
centrifuging the first mixture for 1 to 10 minutes at a speed of 10,000 to 15,000 rpm; and
decanting a supernatant to form the protein lysate.

16. The method of claim 15, further comprising:
adding phosphatase inhibitors and protease inhibitors to the first mixture.

17. The method of claim 1, wherein the antibody cocktail is a single reagent.

18. The method of claim 1, wherein the secondary antibody is conjugated to the primary antibody before the primary antibody and the second primary antibody are conjugated to the target protein.

19. The method of claim 1, wherein the target protein is a mixture of at least two proteins.

20. The method of claim 1, wherein the secondary antibody is conjugated with a signal generator.
